(19) United States
(12) Patent Application Publication
(10) Pub. No.: US 2016/0153973 A1
(43) Pub. Date: Jun. 2, 2016

(54) DEVICE AND METHOD OF RAPID LINKER MEDIATED LABEL-BASED IMMUNOASSAYS

(71) Applicant: Lucas David SMITH, Monticello, IL (US)

(72) Inventor: Lucas David Smith, Monticello, IL (US)

(21) Appl. No.: 14/903,399

(22) PCT Filed: Jul. 9, 2014
(86) PCT No.: PCT/US14/46041

§ 371 Date: Jan. 7, 2016

Related U.S. Application Data
(60) Provisional application No. 61/844,181, filed on Jul. 9, 2013.

(51) Int. Cl. G01N 33/543

(52) U.S. Cl. CPC G01N 33/54306

(57) ABSTRACT

A method and system is provided which uses cleavable linkers to detect an analyte in an immunoassay. The use of linkers in ELISA and similar immunoassay protocols allows for a reduction in wash steps, incubation time, and potential for user error. The linkers create an environment that allows for intramolecular binding kinetics for quickly binding an analyte to two antibodies. The system works with ELISA and other similar protocols, and one embodiment of the invention does not require the binding of antibodies to a solid support. The disclosure also provides a method of making the system of cleavable linkers for use in a variety of immunoassays.

### CROSS-REFERENCE TO RELATED APPLICATIONS

The present application claims the benefit of U.S. Provisional Application No. 61/844,181, filed Jul. 9, 2013, entitled “DEVICE AND METHOD OF RAPID LINKER MEDIATED ANTIGEN DETECTION”.

### BACKGROUND OF THE INVENTION

The present invention generally relates to an immunoassay analyte detection system. More particularly, the present invention relates to a system and method of analyte detection using a unique immune complex to facilitate rapid binding of analytes in immunoassays and related techniques.

The enzyme-linked immunosorbent assay (ELISA) technique is one of the most commonly used methods of immunoassay. Since its inception in 1971, the general state of the art has expanded to include similar techniques such as Fluorescence Linked Immunosorbent Assays (FLISA). Such techniques have been widely used for the detection of macromolecules in a solution. The conventional ELISA uses an enzymatic reaction to produce a quantifiable signal that relates back to the amount of analyte in a sample.

ELISA may be used as a quantitative measure of antibodies or antigens in a sample. If antigens are the analyte, then the typical ELISA uses antibodies bound to a solid support, often a 96-well plate. A sample containing the antigen is then administered to each well. The antibodies bound to the plate will bind with the antigen during an initial step. Typically a “detection” antibody conjugated to a response agent such as an enzyme or chromophore is then added. This is a second antibody then binds to an alternative epitope of the antigen already bound to the first antibody.

Excess (unbound) detection antibodies are then rinsed away from the plate. Following this step, a substrate is added to the wells. The substrate is initially a colorless molecule, which, upon reacting with the enzyme, undergoes a chemical change that produces an observable color or fluorescence. This response is then quantified with a spectrophotometer to determine the amount of absorbance or fluorescence in the solution. This direct or indirect measurement of the detection agent is then utilized to calculate the amount of antigen in the sample.

Typical immunoassays require multiple washing and incubation steps. New techniques have cut down on the number of steps, time taken to complete each step, and potential for user error. Despite these advancements, the conventional ELISA process is still much the same as it was 40 years ago, and requires significant time and user involvement.

### BRIEF SUMMARY OF THE INVENTION

One or more of the embodiments of the present invention provides an immunoassay system and method for analyte detection. The enzyme linked immunosorbent assay (ELISA) is a robust technology for the detection and quantification of antigens in a sample. Since its initial use by Engvall and Perlmann in 1971, ELISA has had an immense impact on academic research, pharmaceutical development, pathogen detection in national defense, food safety, and as an analytical tool for medical diagnostics. Despite the numerous advancements in antibody affinity and analyte detection, the ELISA methodology has remained dependent on the same fundamental steps. The cleavable linker system proposed herein eliminates redundant steps and provides a method by which multiplexed assays are performed faster and with a greater potential for increased automation, accuracy, and sensitivity.

A secondary potential application of the cleavable linker system is in the high throughput research and development market. In recent years the development of DNA microarrays has allowed researchers to measure the expression of thousands of genes based on mRNA expression within cells. This technology is the product of an immense amount of research, and has become an integral part of drug discovery and pharmaceutical development research. While DNA microarrays provide a terrific method of identifying changes in mRNA expression, they are not a direct measurement of protein expression and cannot account for changes in protein expression which are a result of posttranscriptional regulation. The cleavable linker system has the potential to work in conjunction with existing microarray DNA plating and detection methodologies to provide a rapid method of direct protein detection. To conduct this assay, antibodies directed toward desired analytes are conjugated to DNA with a known sequence and adhere to a location on the microarray corresponding with complimentary plated DNA. Samples are then added, washed with a hydrolyzing buffer, and detection completed as normal. This application would be incredibly valuable in pharmaceutical development, and allows for multiplexed protein detection in a single step and in a manner that was largely facilitated by pre-existing technology.

The cleavable linker system is applicable to any immunoassay platform to increase speed and reproducibility. This is of particular importance in medical point of care tests, where assay accuracy and run time have an incredible impact on treatment strategy and success. The cleavable linker system provides a method of eliminating the secondary antibody binding steps in both microfluidic and 96-well plate immunoassays, an improvement which results in a 50% decrease in assay time for fluorescent or color based point of care devices. Further, the surface bound nature of the reported immune complex allows for multiplexed assays to be performed in microfluidic systems, resulting in further decreases in assay incubation time.

The cleavable linker system provides a simpler and more rapid method of medical analyte detection by allowing assays to be conducted without sample contamination by reaction agents. While the vast majority of immunoassays necessarily alter sample composition, all components of the cleavable linker system remain bound during sample incubation. This allows for the potential for subsequent sample analysis even when only a single drop of blood is drawn. This component of the system opens up the potential for secondary sample analysis. Beyond providing the possibility of retrospective analysis and decreasing the need for drawing blood on multiple occasions, this component allows for the potential of result dependent secondary sample analysis, wherein subsequent tests are conducted based on the results of initial analysis.

In recent years, the demand for assays with fewer steps and decreased potential for technician error has resulted in a transition to detection agents that do not require enzymatic activity or substrate development. These have included fluorescent, luminescent, electrochemical, calorimetric, and magnetoelectric detection mechanisms, which only require antigen and detection antibody binding steps prior to signal measurement. While these methods allow for detection following two binding and wash steps, the cleavable linker system allows detection to be achieved with a single binding and wash step.

This cleavable linker system utilizes a more efficient approach to sandwich immunoassays, which eliminates redundant steps to allow for single step immunoassays to be performed in conjunction with a variety of detection agents and assay platforms. This is achieved through the use of a cleavable linker system, which allows for the temporary attachment of surface bound capture antibodies to detection antibodies. When an antigen is added, binding to one antibody results in the binding to the second antibody as a result of the close proximity and intramolecular binding kinetics. This phenomenon results in the formation of a complex where the analyte is bound by both a capture and detection antibody in a manner similar to that formed in the second step of the conventional sandwich ELISA. When this bond has formed, the cleavable linker is no longer needed to secure the detection antibody to the surface, and in turn is cleaved and washed to selectively remove all unbound detection antibodies.

### BRIEF DESCRIPTION OF THE DRAWINGS

FIG. 1 illustrates a system with cleavable linkers for detecting analytes according to an embodiment of the present invention.

FIG. 2 illustrates a system with a cleaved linker for detecting analytes according to an embodiment of the present invention.

FIG. 3 illustrates a system with an antibody bound to a plate and cleavable linkers for detecting analytes according to an embodiment of the present invention.

FIG. 4 illustrates a system with a common linker and cleavable linkers for detecting analytes according to an embodiment of the present invention.

FIG. 5 illustrates a system with cleavable linkers in solution for detecting analytes according to an embodiment of the present invention.

FIG. 6 illustrates a method for binding a linker to a solid support according to an embodiment of the present invention.

FIG. 7 illustrates a method of linker cleavage according to an embodiment of the present invention.

FIG. 8 illustrates a method for binding a linker to an antibody according to an embodiment of the present invention.

FIG. 9 illustrates a flowchart for making a plate with capture and detection antibodies bound to a surface with independent linkers according to an embodiment of the present invention.

FIG. 10 illustrates a flowchart for detecting an analyte with ELISA according to an embodiment of the present invention.

FIG. 11 illustrates a flowchart for detecting an analyte with FLISA according to an embodiment of the present invention.

FIG. 12 illustrates a flowchart for making a capture antibody bound to a plate directly and a detection antibody bound to a plate through a linker according to an embodiment of the present invention.

FIG. 13 illustrates a flowchart for making the capture antibody bound with a linker to a common linker and a detection antibody bound with a second linker to the common linker according to an embodiment of the present invention.

### DETAILED DESCRIPTION OF THE INVENTION

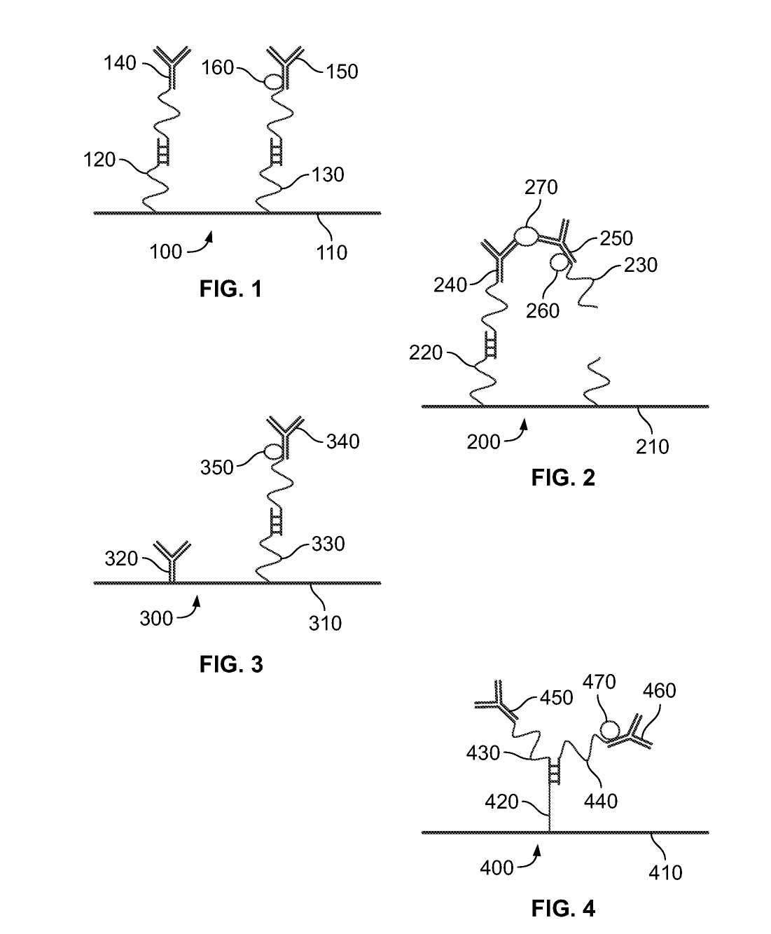

FIG. 1 illustrates a system with cleavable linkers for detecting analytes 100 according to an embodiment of the present invention. The system 100 includes a solid support 110, a linker 120, a cleavable linker 130, a capture antibody 140, a detection antibody 150, and a detection agent 160.

The solid support 110 is chemically bound to the linker 120 and the cleavable linker 130. The capture antibody 140 is chemically bound to the linker 120. The detection antibody is chemically bound to the cleavable linker 130 and the detection agent 160.

In one embodiment, an analyte is introduced to the system 100. The analyte is an antigen with epitopes that bind to a paratrope on both the capture antibody 140 and the detection antibody 150. When the analyte reacts with both paratropes, a complex is formed. Once the complex is formed, the cleavable linker 130 is cleaved. In one embodiment, the cleavable linker 130 is a DNA strand and the strand is cleaved by adding a buffer to the solution which decreases the melting temperature of double stranded DNA interactions with the detection antibody linker. Once the cleavable linker 130 has been cleaved, the sample is washed to remove excess detection antibodies that have not bound to the antigen.

In one embodiment, the detection agent 160 is Horseradish Peroxidase (HRP), and the substrate is 3,3′,5,5 ′-tetramethylbenzidine (TMB). After washing, a substrate is added. The substrate is a colorless substance that the enzyme will react with to form a new molecule that absorbs light, so that the color and intensity of light in the sample is measured to determine the amount of analyte in the sample. The HRP and TMB reaction is known in the prior art as a method of detecting an analyte using Enzyme Linked Immunosorbent Assay (ELISA). After this reaction is complete, the absorbance of the sample at 450 nm is recorded using a spectrometer.

In one alterative embodiment, the capture antibody 140 and detection antibody 150 are replaced by DNA, RNA, or peptide aptamers. Aptamers are oligonucleic acid or peptide molecules that are capable of binding a specific target, similar to antibodies. Aptamers are used in a similar fashion to create the system 100.

In another alternative embodiment, the detection agent 160 is replaced with a fluorophore. This embodiment represents the FLISA protocol. The fluorophore absorbs light from a spectrometer at a certain wavelength and emits light at a second wavelength. This fluorescence can be measured and correlated to the amount of analyte in the sample, thus providing an alternative to adding a substrate to react with the enzyme detection agent 160 as in the ELISA protocol.

Another embodiment, the detection agent 160 bound to the cleavable linker 130 and serves the same function as in the original embodiment.

Another embodiment of the cleavable linker system is the use of a polyethylene glycol (PEG) linker in place of the first linker 120 and the second cleavable linker 140. Another embodiment of the invention is including one of each of the PEG and DNA linkers in the system. These linkers have been shown to achieve similar uses in anchoring a biological molecule to a substrate and having the specific cleavability used in the system.

Another alternative embodiment to enzyme response agents is the use of fluorophores for use in a fluorescence polarization detection method. It is known that fluorophores respond in particular ways to polarized light. In an embodiment that utilizes fluorescence polarization, plane polarized light is utilized in the assay step. The resulting fluorescence of the fluorophore provides information about the binding of the analyte in real time.

Another embodiment is the use of a Fluorescence Resonance Energy Transfer protocol (FRET). In this embodiment, each of the paired antibodies are labeled with paired fluorophores capable of fluorescence resonance energy transfer. When either antibody binds the desired antigen, the proximity of the second antibody leads to complex formation. Binding of the antigen by both antibodies results in the fluorophores being brought within the range of FRET, and the resulting binding kinetics are immediately measured. An alternative embodiment is employed using magnetic or impedance immunoassays, where detection antibodies include a probe, such as silver nanoparticle, whereby probe proximity to a detection surface is measured upon antigen binding.

Yet another alternative embodiment detection mechanism utilizes acceptor beads and donor beads in place of the enzyme 160. The acceptor bead may be attached to the detection antibody or the cleavable linker, or the capture antibody or linker. The donor beads are embedded with photosensitizers that convert oxygen to an excited state singlet oxygen upon illumination at specific wavelengths. The singlet oxygen molecule then transfers energy to the acceptor bead. The acceptor bead then emits light at an observable wavelength. This luminescent oxygen-channeling chemistry is used to perform an amplified luminescent proximity homogeneous assay.

The system 100 allows for carrying out an ELISA procedure with fewer steps than the current preferred method. Conventional ELISAs are performed using a process comprising 4 incubation steps: capture antibody antigen binding, detection antibody binding, incubation with an enzymatic substrate, and enzymatic product colorimetric development. In the prior art, each of these steps requires a 30 minute incubation period as well as 3 labor-intensive wash steps. Each additional step contributes to reagent consumption, technician time required, as well as, the potential for additional human error.

The system 100 addresses these issues through the elimination of the detection antibody binding step, effectively reducing technician time and potential error by up to 33% relative to ELISA assays and 50% relative to fluorescent immunoassays. This is achieved through the use of a cleavable linker, which attaches capture and detection antibodies to form a single detection complex. When an antigen is bound by either antibody, intramolecular binding kinetics results in nearly instantaneous binding of the second antibody. A hydrolyzing wash buffer is then used to cleave the linker, removing all unbound detection antibodies and allowing for detection without an additional binding step.

In the primary embodiment of the cleavable linker system, the binding motifs are paired monoclonal antibodies designed to bind different epitopes on a target antigen, while in other embodiments the antibodies are polyclonal. In some embodiments the antibodies are full, while in others there are partial, comprising of the antigen binding fragment (Fab) or F(ab′)2. In yet other embodiments the binding motif is another protein such as protein A or pathogenic antigens, and in yet other embodiments, the binding motif is a DNA or RNA aptamer.

Upon contact of the sample with the active surface, one antibody binds to a target antigen. Antigen binding to one antibody then drastically facilitates a second binding by the intramolecular nature of the secondary reaction. Upon binding of the antigen to the secondary antibody, the immune complex becomes circularized, and the binding interactions between the two binding fragments results in stabilization of the complex with or without the presence of the linker.

FIG. 2 illustrates a system with a cleaved linker for detecting analytes 200 according to an embodiment of the present invention. The system 200 includes a solid support 210, a linker 220, a cleaved linker 230, a capture antibody 240, a detection antibody 250, a detection agent 260, and an analyte 270. The solid support is chemically bound to the linker 220. The linker 220 is chemically bound to the capture antibody 240. The analyte 270 is chemically bound to the capture antibody 240 and the detection antibody 250. The detection antibody is chemically bound to the cleaved linker 230.

The system 200 is the result of adding an analyte to the system 100. The capture antibody 240 and the detection antibody 250 both bind to the analyte 270 when it is added to the plate. Then, a hydrolyzing buffer is added, and the cleavable linker is cleaved. This results in the system 200. After this, the plate is washed to remove any detection antibodies that have not bound to an analyte. Quantification methods such as ELISA or FLISA are then used to quantify the amount of analyte in the solution.

FIG. 3 illustrates a system with an antibody bound to a plate and cleavable linkers for detecting analytes 300 according to an embodiment of the present invention. The system 300 includes a solid support 310, a capture antibody 320, a cleavable linker 330, a detection antibody 340, and a detection agent 350.

The solid support 310 is chemically bound to the capture antibody 320 and the cleavable linker 330. The cleavable linker 330 is chemically bound to the detection antibody 340. The detection antibody 340 is chemically bound to the detection agent 350.

In one embodiment, an analyte is introduced to the system 300. In the preferred embodiment, the analyte is an antigen with epitopes that bind to a paratrope on both the capture antibody 320 and the detection antibody 340. When the analyte reacts with both paratropes a complex is formed. Once the complex is formed, the cleavable linker 330 is cleaved. In one embodiment, the cleavable linker 330 is a DNA strand and the strand is cleaved by adding a buffer to the solution. Once the cleavable linker 330 has been cleaved, the sample is washed to remove excess detection antibodies that have not bound to the antigen.

In one embodiment, the detection agent 350 is Horseradish Peroxidase (HRP), and the substrate is 3,3′,5,5′-tetramethylbenzidine (TMB). After washing, a substrate is added. The substrate is a colorless substance that the enzyme will react with to form a new molecule that absorbs light, so that the color and intensity of light in the sample is measured to determine the amount of analyte in the sample. The HRP and TMB reaction is known in the prior art as a method of detecting an analyte using Enzyme Linked Immunosorbent Assay (ELISA). After this reaction is complete, the absorbance of the sample at 450 nm is recorded using a spectrometer.

Similar to the invention of FIG. 1, the system 300 reduces the time and potential for error in ELISA and Fluorescence immunoassay techniques.

FIG. 4 illustrates a system with a common linker and cleavable linkers for detecting analytes 400 according to an embodiment of the present invention. The system 400 includes a solid support 410, a common linker 420, a linker 430, a cleavable linker 440, a capture antibody 450, a detection antibody 460, and a detection agent 470.

The solid support 410 is chemically bound to the common linker 420. The common linker 420 is chemically bound to the linker 430 and the cleavable linker 440. The linker 430 is chemically bound to the capture antibody 450. The cleavable linker is chemically bound to the detection antibody 460. The detection antibody 460 is chemically bound to the enzyme 470.

In one embodiment, an analyte is introduced to the system 400. In the preferred embodiment, the analyte is an antigen with epitopes that bind to a paratrope on both the capture antibody 450 and the detection antibody 460. When the analyte reacts with both paratropes a complex is formed. Once the complex is formed, the cleavable linker 440 is cleaved. In one embodiment, the common linker 420 is a double strand of DNA and the linker 430 is a single strand branch of the common linker 420. The cleavable linker 440 is also a single DNA strand branching off the common linker 420. The cleavable linker 440 is cleaved by adding a buffer to the solution. Once the cleavable linker 440 has been cleaved, the sample is washed to remove excess detection antibodies that have not bound to the antigen.

In one embodiment, the detection agent 470 is Horseradish Peroxidase (HRP), and the substrate is 3,3′,5,5′-tetramethylbenzidine (TMB). After washing, a substrate is added. The substrate is a colorless substance that the enzyme will react with to form a new molecule that absorbs light, so that the color and intensity of light in the sample is measured to determine the amount of analyte in the sample. The HRP and TMB reaction is known in the prior art as a method of detecting an analyte using Enzyme Linked Immunosorbent Assay (ELISA). After this reaction is complete, the absorbance of the sample at 450 nm is recorded using a spectrometer.

Similar to previous figures, the system 400 reduces steps and potential for user error in ELISA and FLISA protocols.

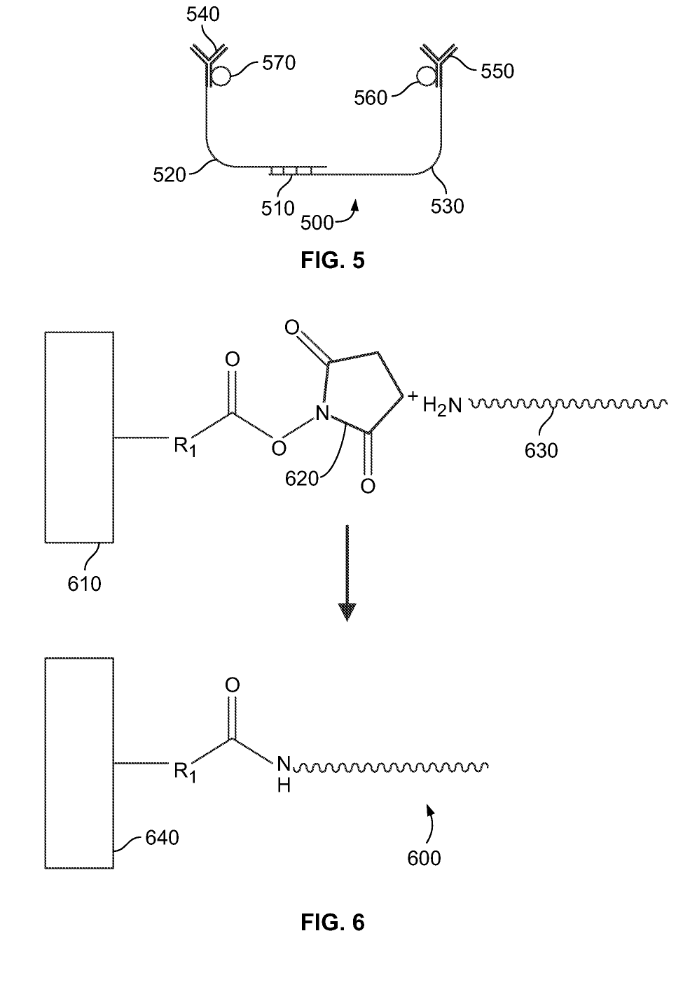

FIG. 5 illustrates a system with cleavable linkers in solution for detecting analytes 500 according to an embodiment of the present invention. The system 500 includes a common cleavable linker 510, a first linker 520, a second linker 530, a capture antibody 540, a detection antibody 550, a first fluorophore 560, and a second fluorophore 570.

In one embodiment, the common cleavable linker 510 is a double strand of DNA. The first linker 520 is a single strand of that common cleavable linker 510 that is chemically bound to the common cleavable linker 510. The second linker 530 is also a single strand of DNA chemically bound to the common linker 510. The capture antibody 540 is chemically bound to the second linker 530 and the first fluorophore 560. The detection antibody 550 is chemically bound to the first linker 520 and the second fluorophore 570.

When an analyte is introduced to the system 500, the capture antibody 540 and the detection antibody 550 bind to the analyte. In the preferred embodiment, the analyte is an antigen. Once the analyte is bound, the common cleavable linker 510 is cleaved by adding a buffer to the solution. The sample then undergoes a Fluorescence Resonance Energy Transfer (FRET) protocol wherein the sample is irradiated with light of a particular wavelength, which is absorbed by the first fluorophore 560. The energy absorbed by the first fluorophore 560, and transferred to a second fluorophore 570. The energy is then emitted by the second fluorophore 570 at a second wavelength, which is distinct from the fluorescence emission wavelength of the first fluorophore. This signal is measured by a spectrometer. In an alternative embodiment, the common cleavable linker 510 is cleaved by adding a buffer to the solution following analyte binding, and measured subsequent to linker cleavage.

In this embodiment, there is no need to wash the solution to get rid of excess antibodies. The immunocomplexes that have bound an antigen will remain bound and in intramolecular proximity. This allows for maintained intramolecular proximity of the fluorophore on the capture antibody and the fluorophore on the detection antibody. This proximity allows for the FRET protocol to work. For those complexes that have not bound an antigen, initial distance or cleavage of the common linker 510 results in a distance too great for the fluorophores to effectively transfer the fluorescence energy, thus resulting in negligible observable signal in the absence of antigen.

The system 500 is made by binding fluorophores to a capture antibody and a detection antibody separately. The capture antibody and detection antibody are capable of binding alternative epitopes of one antigen. The antibodies are then bound to a linker, as shown in FIG. 5. In the case of a DNA linker, the linker on the capture antibody has the conjugate base pairs of the linker on the detection antibody. When the capture antibody-linker-fluorophore complex and detection antibody-linker-fluorophore complex are added to the same solution, the Watson-Crick base pairs will spontaneously form a chemical bond, creating the system 500.

FIG. 6 illustrates a method for binding a linker to a solid support 600 according to an embodiment of the present invention. The method 600 includes a solid support 610, a N-oxysuccinimide moiety 620, an amide linker 630 in pre-binding linker chemistry, and a solid support with linker 640 in post-binding linking chemistry.

The solid support 610 is chemically bound to the N-oxysuccinimide moiety 620. The amide linker 630 reacts with the N-oxysuccinimide moiety 620 to form the amide in solid support with linker 640. In operation, the reaction in the method 600 is used to bind a linker to a solid support.

There are a variety of possible methods for attaching the linker to the solid support. One alternative embodiment includes using an azide functional group attached to the solid support. An alkynyl-modified oligonucleotide reacts with the azide in what is known as the “Click” reaction to form a bond between the linker and the plate. Another alternative embodiment includes adding a biotinylated oligonucleotide to a solid support bound to streptavidin to form the bond. Yet another embodiment includes reacting a sulfhydryl-modified linker with a solid support with an iodoacetyl moiety. The iodoacetyl moiety is capable of immobilizing ligands with sulfhydryl groups to form the bond between linker and solid support.

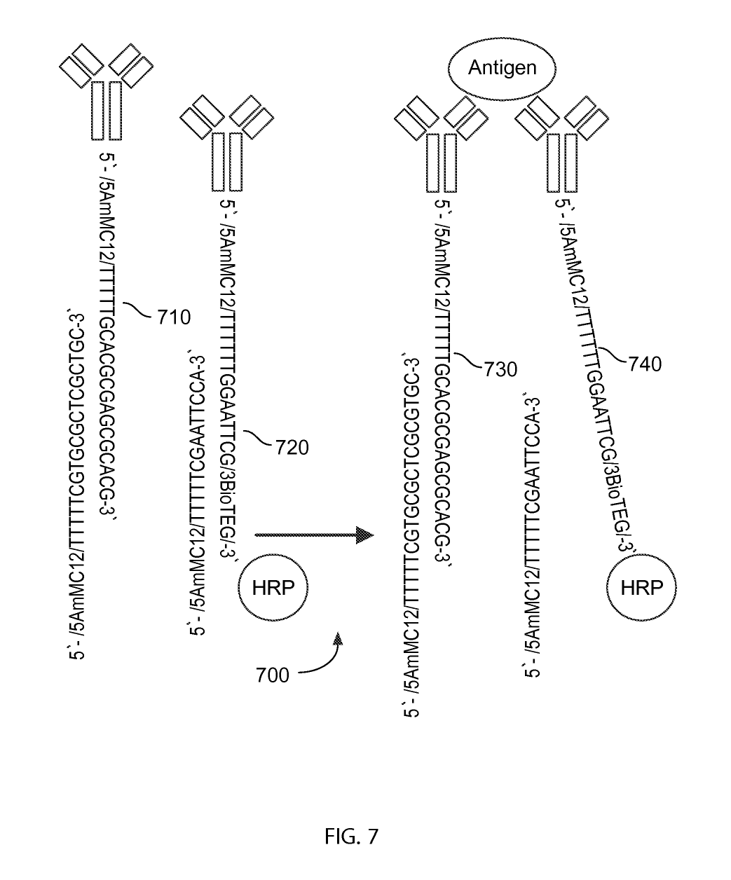

FIG. 7 illustrates a method of linker cleavage 700 according to an embodiment of the present invention. The method 700 includes a capture antibody with DNA linker 710, a detection antibody with DNA linker and enzyme 720, a capture antibody with uncleaved DNA linker 730, and a detection antibody with cleaved DNA linker and enzyme 740.

In operation, the detection antibody with DNA linker and enzyme 720 is cleaved selectively using a buffer such as Tris-buffered Saline (50 mM Tris, 150 mM sodium chloride, 30% methanol, pH 7.5) containing 0.5% Tween-20 (TBST). The buffer solution selectively cleaves the detection antibody with DNA linker and enzyme 720 but leaves the capture antibody with uncleaved DNA linker 730 intact.

The bottom half of the linker is the oligonucleotide that is deposited on the plate as shown in the method 600. The top half, a result of the antibody-oligonucleotide conjugation reaction as shown in the method 800, is added to the existing plate, as is further detailed in step 950 of flowchart 900. When the antibody conjugated oligonucleotide is added to the oligonucleotide coated plate, the Watson-Crick base pairs react to form the capture antibody with DNA linker 710 and detection antibody with DNA linker and enzyme 720.

In one embodiment, the oligonucleotide sequences for each linker are as follows:

### Capture Plate Binding Oligonucleotide

a. 5′-/5AmMC12/TTTTTCGTGCGCTCGCGTGC-3′
b. Maximum ΔG=−46.12 kcal/mole
c. Melting Temp.=61.7 C (0.25 uM, 50 mM NaCl)
d. Homodimer ΔG=−10.36 kcal/mole
e. Heterodimer ΔG=−8.47 kcal/mole

### Capture Antibody Binding Oligonucleotide

a. 5′-/5AmMC12/TTTTTGCACGCGAGCGCACG-3′
b. Maximum ΔG=−46.12 kcal/mole
c. Melting Temp.=61.7 C (0.25 uM, 50 mM NaCl)
d. Homodimer ΔG=−5.19 kcal/mole
e. Heterodimer ΔG=−5.19 kcal/mole

### Detection Plate Binding Oligonucleotide

a. 5′-/5AmMC12/TTTTTTTTTTTTTTTTTTTTCGAATTCCA-3′
b. Maximum ΔG=−17.15 kcal/mole
c. Melting Temp.=21.8 C (0.25 uM, 50 mM NaCl)
d. Homodimer ΔG=−8.51 kcal/mole
e. Heterodimer ΔG=-7.13 kcal/mole

### Detection Antibody Binding Oligonucleotide

a. 5-/5AmMC12/TTTTTTTTTTTTTTTTTTTTTTTTTTGCTGGAA TTCGTCG/3TYE563/-3′
b. Maximum ΔG=−17.15 kcal/mole
c. Melting Temp.=21.8 C (0.25 uM, 50 mM NaCl)
d. Homodimer ΔG=−8.51 kcal/mole
e. Heterodimer ΔG=−7.13 kcal/mole

Though some detection mechanisms in the prior art do not use sample washing, the most sensitive detection systems require removal of unbound detection antibodies. In order to completely remove all unbound detection antibodies, the DNA linker used must be cleaved and removed without disturbing the bound complexes. One of the most well studied methods by which this is achieved is through the use of single stranded DNA-antibody conjugates with a complimentary region designed to melt upon the addition of heated wash buffers. An alternative strategy is to use wash buffers containing compounds that facilitate DNA melting through the use of gradients in salt concentration, polyols, alcohols, urea, formamide, pH, or alternative solvents. Another strategy utilizes controlled wash buffer flow rate that causes shear stress induced DNA melting.

While DNA denaturation provides a well-studied methodology for controlling linker dissociation, covalently bound complexes frequently provide drastic increases in stability. Though DNA cleavage is no longer controlled through melting, there are a myriad of alternative mechanisms by which DNA hydrolysis is still achieved. The most common of these mechanisms is the use endonucleases, or enzymes that cleave DNA phosphodiester bonds at specific sites within the DNA. Endonucleases are utilized in many molecular biology techniques, and have been engineered to allow for DNA cleavage in a matter of minutes. An alternative method utilizes photo cleavable spacers, which are designed between DNA bases, allowing for controlled cleavage upon exposure to UV light. Yet another alternative involves the use of DNA containing chemically cleavable regions such as di-sulphorde or 1,2-bis(alkylthio) ethane moieties, which are cleaved respectively through reduction with dithiothreitol (DTT) or using singlet oxygen species generated with light in the presence of a singlet oxygen photosensitizer (PS). In either case, chemical agents are contained within a wash buffer, and cleavage occurs in tandem with detection antibody removal. Beyond increasing effective plate binding capacity, the use of DNA allows for economical bi-functional modification, as well as, a variety of commercially available and well established cleavage mechanisms.

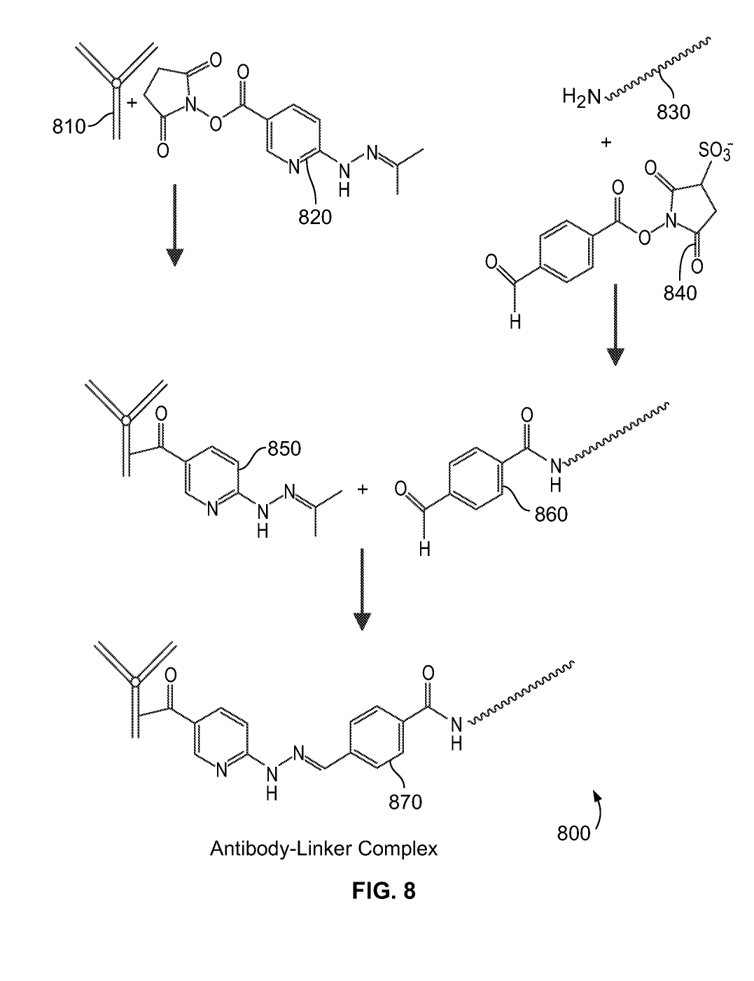

FIG. 8 illustrates a method for binding a linker to an antibody 800 according to an embodiment of the present invention. The method 800 includes an antibody 810, a S-HyNic (succinimidyl-6-hydrazino-nicotinamide) crosslinker 820, an amine linker 830, a S-4FB (N-succinimidyl-4-formylbenzamide) crosslinker 840, a crosslinker functionalized antibody 850, a crosslinker functionalized linker 860, and an antibody-linker complex 870.

In operation, the S-HyNic crosslinker 820 reacts with the antibody 810 to form the crosslinker functionalized antibody 850. The amine linker 830 reacts with the S-4FB crosslinker 840 to form the crosslinker functionalized linker 860. The crosslinker functionalized antibody 850 and the crosslinker functionalized linker 860 react to form the antibody-linker complex 870.

The method 800 is one embodiment for binding a linker to an antibody. The reaction is used to form the capture antibody bound to a linker as shown in FIGS. 1-4 or the detection antibody bound to a linker as shown in FIGS. 1-4.

In order to assemble the preferred embodiment of the complex, the DNA is conjugated to antibodies in a stable fashion, which does not result in a loss of binding activity. Antibody-oligomer conjugation is an area which has been thoroughly studied for applications including immuno-PCR, as fusion proteins for cellular delivery of oligonucleotides, for multiplexed protein analysis of single cells, and even for the construction of dual specificity antibodies. There are also a large number of independent conjugation techniques for attaching DNA to nucleotides. The most common of these techniques utilize amine-modified DNA modified with at Sulfo-S-4FB (N-succinimidyl-4-formylbenzamide) linker as well as an antibody modified using the S-HyNic (succinimidyl-4-formylbenzamide) linker. When these components are combined, a stable covalent conjugate is formed with a 4FB (4-formylbenzamide) linker connecting the DNA and the antibody. In an alternative method, aldehyde-modified oligonucleotides react with hydrazine-modified antibodies forming a conjugate containing a hydrazone bond. With either method of conjugation, it has been shown that antibodies with similar activity nearly identical to that of the unconjugated antibody are produced.

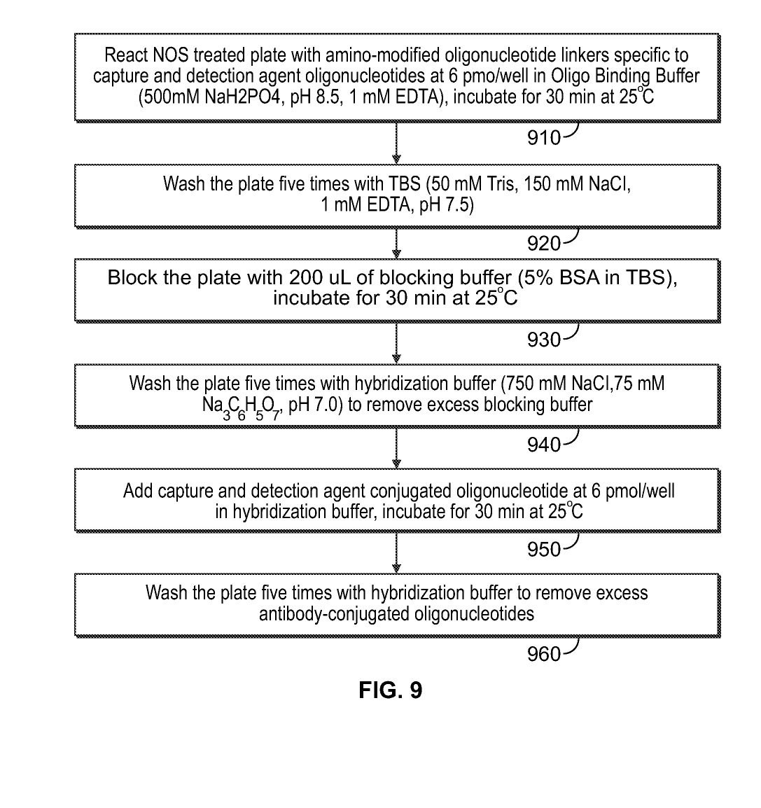

FIG. 9 illustrates a flowchart for making a plate with bound linkers capture antibodies and detection antibodies 900 according to an embodiment of the present invention. As shown in FIG. 9, the first step 910 in flowchart 900 is to add an amino-modified C12 linker-armed oligonucleotide to a commercially available N-oxysuccinimide (NOS) treated plate. The oligonucleotide linkers are specific to the oligonucleotides bound to the capture and detection agents so that binding will occur when they are later introduced. The amine on the oligonucleotide reacts with the NOS group to form an amide bond. The reaction is performed in Oligo Binding Buffer (500 mM NaH₂PO₄, pH 8.5, 1 mM EDTA). The amino-modified C12 linker-armed oligonucleotide is preferably at a concentration of 6 pmol/well. This reaction is further detailed in FIG. 5. Then in step 920, the plate is washed five times with TBS (50 mM Tris, 150 mM NaCl, pH 7.5). Step 920 is used to remove any oligonucleotide that has not bound to the plate.

Then, in step 930, unreacted NOS ester groups on the plate are blocked with 5% Bovine Serum Albumen (BSA). The plate is then incubated at 25° C. for 30 minutes, and the excess BSA fluid is decanted. The plate is then washed five times with hybridization buffer (750 mM NaCl, 75 mM Na₃C₆H₅O₇, pH 7.0) in step 940. The capture and detection antibodies with attached oligonucleotide linkers are then added and allowed to react with the linkers currently attached to the plate in step 950, and incubated for 30 minutes at 25° C. Finally, in step 960, the plate is washed five times with hybridization buffer. After completing these steps, the system 100 is formed.

In addition to the benefits of performing immunoassays in a single step, the use of linker mediated binding provides drastically increased antibody surface binding activity. In conventional antibody surface attachment, antibodies are non-specifically bound to a surface. This nonspecific binding results in a substantial decrease in the concentration of bound antibodies as well as an even more pronounced decrease in the number of productively oriented antibodies able to bind antigens. Experiments have shown that nonspecific binding results in nearly 90% of antibodies being adhered in non-productive orientations. While the use of specific binding agents intended to bind the conserved region of the antibody increase antibody activity nearly 9 fold, the overall concentration of antibodies remains dependent on the surface area. As with the specific binding agents, extended linkers have been shown to drastically increase antibody activity when bound to an antibody nucleotide binding site. This conjugation method has been shown to increases activity 94-fold and 674-fold, respectively, relative to amine reactive maleic anhydride and physical absorption based methods, as well as, a 3.6-fold and 280-fold increase in detection efficiency.

The preferred complex utilizes linkers which provide increased potential concentration without restricting conformation and inhibiting antibody binding. DNA is used as a linker to provide a flexible, easily synthesized, cleavable agent, which is attached to both antibodies and binding surfaces. While antibodies adhered directly and using specific binding agents are limited to a concentration of 400-500 ng/cm² (2.7-3.3 pmol/cm²), biotin conjugated or covalently attached DNA is plated at an optimum concentration nearly 10 times higher (25-33.2 pmol). Beyond the benefits of linker-mediated attachment, the use of linkers of varying length is employed to reduce steric hindrance between antibodies to further increase the antibody surface concentration. Together, these factors allow linker mediated immune attachment to increase antibody activity and effective concentration, allowing for increased assay detection range and sensitivity.

In an alternative embodiment, the capture agent and detection agent oligonucleotides are bound to an amino-modified linker. The amino-modified linker is then added to an NOS treated plate to form the system.

In another alternative embodiment, the amino-modified oligonucleotides are reacted with the NOS treated plate. Then, the second amino-modified oligonucleotide is added, and the Watson-Crick base pairs react to form the linker. Then, the capture and detection agents are added, and the second amino-modified oligonucleotide reacts with the capture and detection agents to form the system.

In another alternative embodiment, the capture agent oligonucleotides are added to the system first separately from the detection agent oligonucleotides. Then the detection agent oligonucleotides are added. In another alternative, the detection agent oligonucleotides are added first, and the capture agent oligonucleotides are added second separately.

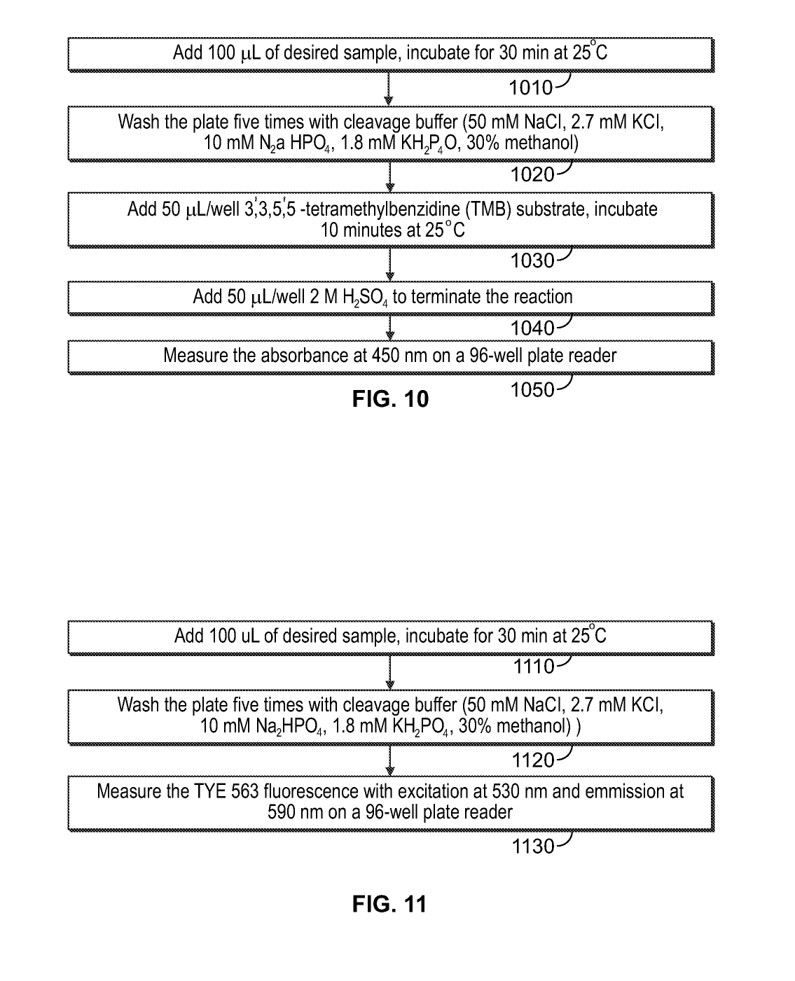

FIG. 10 illustrates a flowchart for detecting an analyte with ELISA 1000 according to an embodiment of the present invention. The flowchart 1000 is used with any of the systems 100, 300 or 400. First, in step 1010, the sample to be tested is added to the 96-well plate containing the antigen detection complex of claim 1. The plate is then incubated at 25° C. for 30 minutes in step 1010. Then, in step 1020, the plate is washed five times with the cleavage buffer (50 mM NaCl, 2.7 mM KCl, 10 mM Na₂HPO₄, 1.8 mM KH₂PO₄, 30% methanol). Next, in step 1030, the 3,3′, 5,5 ′-tetramethylbenzidine (TMB) substrate is added and the plate is incubated for 10 minutes at 25° C.

In step 1040, 2 M sulfuric acid is added (50 μL/well) to terminate the reaction. Finally, the absorbance of each well at 450 nm is measured using a 96-well plate reader in step 1050. The absorbance is used to calculate the quantity of analyte in each well of the plate.

Following the wash of the active analyte binding region and removal of the unbound antibody-detection agent complexes, the analyte detection system is used to identify the concentration of each of the target analytes and in some instances to provide more direct diagnostic information through the analysis of analyte profile. In these embodiments, the detection of the agents remaining bound is conducted at specific reading areas where binding agents directed to a known analyte are adhered.

In the preferred embodiment of the cleavable linker system, a spectrophotometer or fluorometer is used to measure the absorbance or fluorescence at the analyte binding surfaces. In this embodiment, the detection molecule is one capable of absorbing or fluorescing, and an optical window is used to allow for the passage of light at wavelengths corresponding to the fluorophore being used. Detection of analyte binding occurs through the adhesion of optically active probes when the analyte is present. The detection limit of ELISA systems are dependent on the enzyme and substrate used.

FIG. 11 illustrates a flowchart for detecting an analyte with FLISA 1100 according to an embodiment of the present invention. The flowchart 1100 is used with any of the systems 100, 300 or 400. First, in step 1110, the sample to be tested is added to the 96-well plate containing the antigen detection complex of claim 1, wherein the detection agent 160 is replaced by a fluorophore. The plate is incubated at 25° C. for 30 minutes. Then, in step 1120, the plate is washed five times with the cleavage buffer (50 mM NaCl, 2.7 mM KCl, 10 mM Na₂HPO₄, 1.8 mM KH₂PO₄, 30% methanol). Finally, the TYE 573 fluorescence with excitation at 530 nm and emission at 590 nm on a 96-well plate reader is recorded to determine the amount of analyte present in each well in step 1130. FLISA detection limits are dependent on the fluorophore used, though the system has shown a detection limit at around 60 pg/mL.

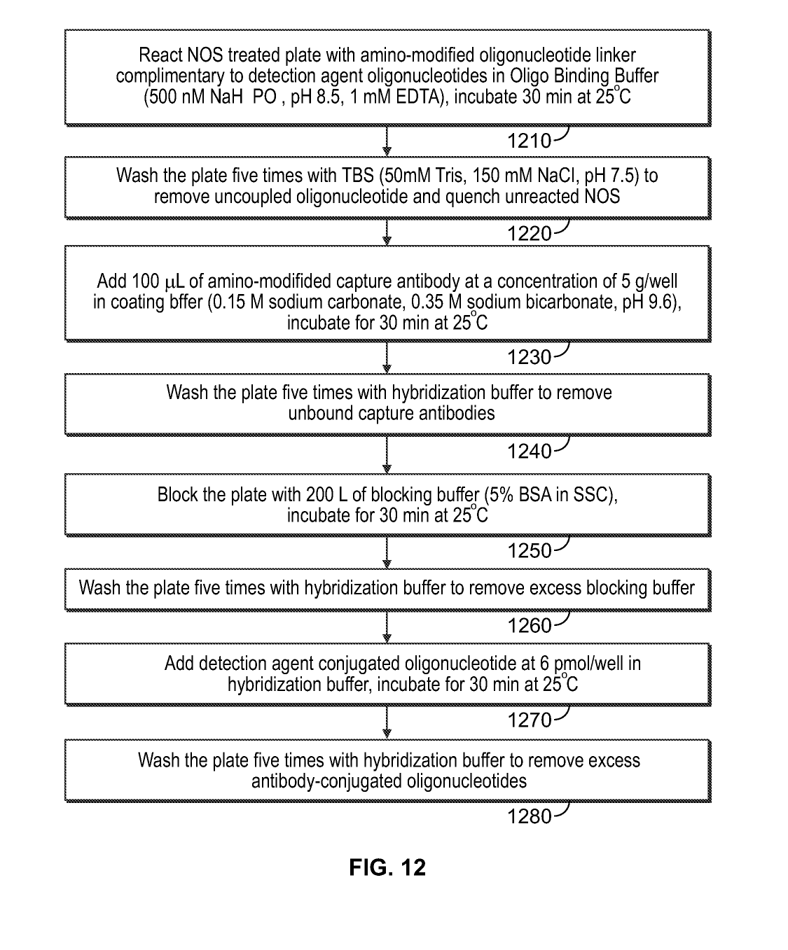

FIG. 12 illustrates a flowchart for making a capture antibody bound to a plate and a detection antibody bound to a plate through a linker 1200 according to an embodiment of the present invention. First, in step 1210, amino-modified oligonucleotide linkers that have complimentary base pairs to detection agent oligonucleotides are bound to an NOS treated plate, and incubated for 30 min at 25° C. In step 1220, the plate is washed five times with TBS. Then 100 μL of a capture antibody solution at a concentration of 6 μg/well in coating buffer (0.15 M sodium carbonate, 0.35 M sodium bicarbonate at a pH of 9.6) is added to the plate in step 1230.

Then in step 1240, the plate is washed five times with hybridization buffer to remove any unbound capture antibodies. In step 1250, the plate is treated with blocking buffer (5% BSA in TBS) to block nonspecific binding. In step 1260, the plate is washed with hybridization buffer to remove any excess blocking buffer. Then, in step 1270, detection agent conjugated oligonucleotides with complimentary base pairs to the plate-bound linkers are added at a concentration of 6 pmol/well in hybridization buffer and incubated for 30 min Finally the plate is washed in step 1280 to remove unbound detection agent oligonucleotides. The result of the flowchart 1200 is shown in FIG. 3.

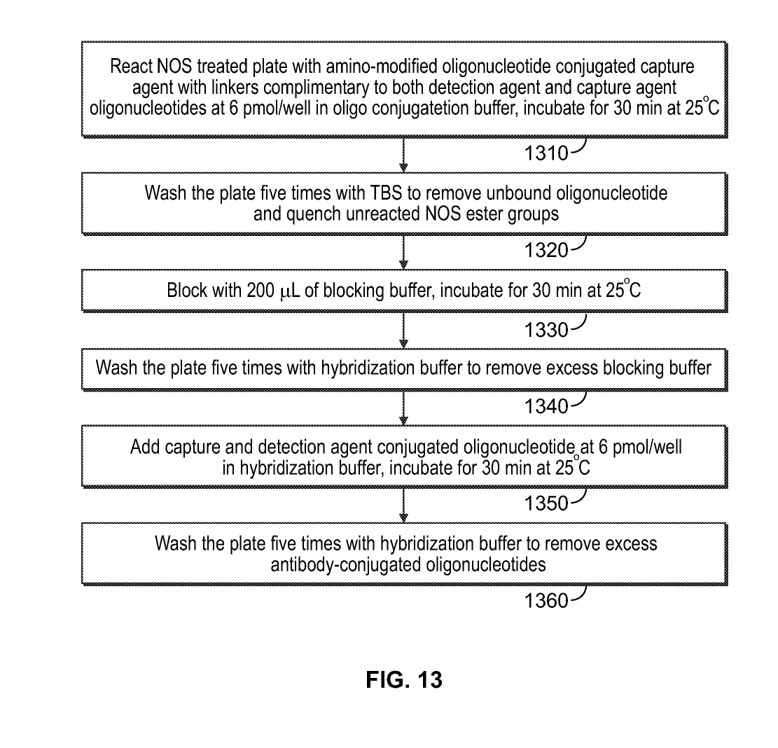

FIG. 13 illustrates a flowchart for making the capture antibody bound with a linker to a common linker and a detection antibody bound with a second linker to the same common linker 1300 according to an embodiment of the present invention. In step 1310, an NOS treated plate is reacted with an amino-modified oligonucleotide with a region with base pairs complimentary to the capture agent and detection agent conjugated oligonucleotides, and incubated for 30 min at 25° C. Then in step 1320, the plate is washed with TBS to remove unbound oligonucleotides and quench any unreacted NOS ester groups. Next, blocking buffer (5% BSA in TBS) is used to block the plate in step 1330. Then the plate is washed to remove excess blocking buffer from the plate in step 1340. Then, detection agent and capture agent conjugated oligonucleotides with base pair complimentary to common linker capture agent oligonucleotides are added to the plate in hybridization buffer in at a concentration of 6 pmol/well in step 1350. These oligonucleotides react with the double stranded oligonucleotide linker to form Watson-Crick base pairs and complete the linker system of FIG. 4. Finally, the plate is washed to remove excess antibody-conjugated oligonucleotides in step 1360. The result of the flowchart 1300 is shown in FIG. 4.

In one system-wide alterative embodiment, the capture and detection antibodies are replaced by DNA, RNA, or peptide aptamers. Aptamers are oligonucleic acid or peptide molecules that are capable of binding a specific target, similar to antibodies. Aptamers are used in a similar fashion to antibodies.

In another alternative embodiment, the enzyme is replaced with a fluorophore. This embodiment represents the FLISA protocol. The fluorophore absorbs light of a particular wavelength and then emits light at a second wavelength. This wavelength is then measured to calculate the amount of analyte in a sample, thus providing an alternative to adding a substrate to react with the enzyme in the ELISA protocol.

Another alternative embodiment is for the enzyme or the fluorophore to be bound to the second cleavable linker. This alternative is further illustrated in FIG. 7. The enzyme is bound to the linker and serves the same function of converting a colorless substrate into a colored compound. In the alternative embodiment, it is a fluorophore for absorbing and emitting light at particular wavelengths.

Another embodiment of the cleavable linker system is the use of a polyethylene glycol (PEG) linker in place of the DNA strand linker. There is one each of a PEG and DNA linker in the system in an alternative embodiment. These linkers have been shown to achieve similar uses in anchoring a biological molecule to a substrate and having the specific cleavability used in the system.

One other embodiment of the invention is the manner in which the linker is cleaved. One of the most well studied methods by which this is achieved is through the use of single stranded DNA-antibody conjugates with a complimentary region designed to melt upon the addition of heated wash buffers. An alternative strategy is to use wash buffers containing compounds that facilitate DNA melting through the use of gradients in salt concentration, urea, formamide, or pH. Another strategy utilizes controlled wash buffer flow rate that causes shear stress induced DNA melting.

While DNA denaturation provides a well-studied methodology for controlling linker dissociation, covalently bound complexes frequently provide drastic increases in stability. Though DNA cleavage is no longer controlled through melting, there are a myriad of alternative mechanisms by which DNA hydrolysis is achieved. The most common of these mechanisms is the use endonucleases, or enzymes that cleave DNA phosphodiester bonds at specific sites within the DNA. Endonucleases are utilized in many molecular biology techniques, and have been engineered to allow for DNA cleavage in a matter of minutes. An alternative method utilizes photo cleavable spacers, which are designed between DNA bases, allowing for controlled cleavage upon exposure to UV light. Yet another alternative embodiment involves the use of DNA containing chemically cleavable regions such as di-sulphorde or 1,2-bis(alkylthio) ethane moieties, which are cleaved respectively through reduction with DTT or using singlet oxygen species generated with light in the presence of a singlet oxygen photosensitizer (PS). In either embodiment, chemical agents are contained within a wash buffer, and cleavage occurs in tandem with detection antibody removal. Beyond increasing effective plate binding capacity, the use of DNA allows for economical bi-functional modification, as well as, a variety of commercially available and well established cleavage mechanisms.

Another alternative embodiment to enzyme response agents is the use of fluorophores for use in a fluorescence polarization detection scheme. It is known that fluorophores respond in particular ways to polarized light. In an embodiment that utilizes fluorophores for fluorescence polarization, plane polarized light is utilized in the assay step. The resulting fluorescence of the fluorophore provides information about the binding of the analyte in real time.

One embodiment of the invention uses Fluorescence Resonance Energy Transfer (FRET) to quantify antigen concentration in real time without the need for a wash step. In this system, each of the paired antibodies are labeled with paired flurophores capable of fluorescence resonance energy transfer. When either antibody binds the desired antigen, the proximity of the second antibody as a result of being bound to the plate leads to rapid complex formation. Binding of the antigen by both antibodies results in the fluorophores being brought within the range of FRET, a binding interaction interaction which is then immediately measured. An alternative embodiment is employed using magnetic or impedance immunoassays, where detection antibodies include a probe, such as silver nanoparticle, whereby probe proximity to a detection surface is measured upon antigen binding.

Yet another alternative embodiment detection mechanism utilizes acceptor beads and donor beads in place of fluorophores. The donor beads are embedded with photosensitizers that convert oxygen to an excited state singlet oxygen upon illumination at specific wavelengths. The singlet oxygen molecule then transfers energy to the acceptor bead. The fluorophores in the acceptor bead then emit light at an observable wavelength. This luminescent oxygen-channeling chemistry is used to perform an amplified luminescent proximity homogeneous assay.

The systems and methods of cleavable linkers for use in immunoassays allow for reduced time spent running immunoassays and reduce the risk of user error. This is accomplished by removing necessary steps currently performed in an typical immunoassay. The systems and methods are also widely adaptable for use with varied response agents such as enzymes or fluorophores, and varied linkers such as DNA or PEG.

While particular elements, embodiments, and applications of the present invention have been shown and described, it is understood that the invention is not limited thereto because modifications may be made by those skilled in the art, particularly in light of the foregoing teaching. It is therefore contemplated by the appended claims to cover such modifications and incorporate those features which come within the spirit and scope of the invention.

1. A solid support,
wherein a capture agent is bound to said solid support by a first linker,
wherein said capture agent is a molecule that chemically binds to an analyte,
wherein a detection agent is bound to said solid support by a second linker,
wherein said detection agent is a molecule that chemically binds to said analyte,
wherein said detection agent is also chemically bound to a response agent.

2. The solid support of claim 1, wherein said capture agent is a protein.

3. The solid support of claim 1, wherein said detection agent is a protein.

4. The solid support of claim 1, wherein said capture agent is an antibody.

5. The solid support of claim 1, wherein said detection agent is an antibody.

6. The solid support of claim 1, wherein said capture agent is an aptamer.

7. The solid support of claim 1, wherein said detection agent is an aptamer.

8. The solid support of claim 1, wherein said response agent is an enzyme.

9. The solid support of claim 1, wherein said response agent is a fluorophore.

10. The solid support of claim 1, wherein said solid support is a multi-well plate.

11. The solid support of claim 1, wherein said solid support is a microfluidic chip.

12. The solid support of claim 1, wherein said solid support is a lateral flow strip.

13. The solid support of claim 1, wherein said first linker is a nucleic acid.

14. The solid support of claim 1, wherein said second linker is a nucleic acid.

15. The solid support of claim 1, wherein said first linker is a polyethylene glycol polymer.

16. The solid support of claim 1, wherein said second linker is a polyethylene glycol polymer.

17. The solid support of claim 1, wherein said first linker is an amide bond.

18. The solid support of claim 1, wherein a second response agent is bound to said capture antibody.

19. The solid support of claim 1, wherein a second response agent is bound to said first linker.

20. An analyte detection complex, wherein said analyte detection complex includes:
a capture agent,
wherein said capture agent is a molecule that chemically binds to an analyte,
wherein said capture agent is chemically bound to a first response agent; and
a detection agent,
wherein said detection agent is a molecule that chemically binds to an analyte,
wherein said detection agent is chemically bound to a second response agent,
wherein said capture agent and said detection agent are chemically bound by a linker,
wherein when a fluid including an analyte is introduced to said detection complex, said capture agent and said detection agent chemically bind to said analyte,
wherein when said linker is cleaved, said capture agent and said detection agent remain bound to said analyte.

21. The analyte detection complex of claim 20, wherein said capture agent is a protein.

22. The analyte detection complex of claim 20, wherein said detection agent is a protein.

23. The analyte detection complex of claim 20, wherein said capture agent is an antibody.

24. The analyte detection complex of claim 20, wherein said detection agent is an antibody.

25. The analyte detection complex of claim 20, wherein said capture agent is an aptamer.

26. The analyte detection complex of claim 20, wherein said detection agent is an aptamer.

27. The analyte detection complex of claim 20, wherein said first response agent is a fluorophore.

28. The analyte detection complex of claim 20, wherein said second response agent is a fluorophore.

29. The analyte detection complex of claim 20, wherein said linker is a nucleic acid.

30. The analyte detection complex of claim 20, wherein said linker is a polyethylene glycol polymer.

31. A method for making a solid support system for detecting analytes, said method including:
reacting a solid support with a linker-capture agent complex to form a linker coated solid support,
wherein said linker-capture agent complex includes a first linker and a capture agent,
wherein said first linker is chemically bound to said capture agent,
wherein said solid support includes a first binding moiety,
wherein said first linker includes a second binding moiety,
wherein first binding moiety and said second binding moiety form a chemical bond,
reacting said linker coated solid support with a linker-detection agent complex to form a solid support system for detecting analytes,
wherein said linker-detection agent complex includes a second linker, a detection agent, and a response agent,
wherein said detection agent is chemically bound to said second linker,
wherein said detection agent is chemically bound to said response agent,
wherein said first linker and said second linker have distinct chemical structures,
wherein said second linker includes a third binding moiety,
wherein said third binding moiety and said first binding moiety form a chemical bond.

24. The method of claim 31, wherein said solid support is a multi-well plate.

25. The method of claim 31, wherein said first linker is a nucleic acid.

26. The method of claim 31, wherein said second linker is a nucleic acid.

27. The method of claim 31, wherein said first linker is a polyethylene glycol polymer.

28. The method of claim 31, wherein said second linker is a polyethylene glycol polymer.

29. The method of claim 31, wherein said first linker is an amide bond.

30. The method of claim 31, wherein said first binding moiety is an N-oxysuccinimide ester.

31. The method of claim 31, wherein said second binding moiety is an amino group.

32. The method of claim 31, wherein said third binding moiety is an amino group.

33. The method of claim 31, wherein said capture agent is an antibody.

34. The method of claim 31, wherein said capture agent is an aptamer.

35. The method of claim 31, wherein said detection agent is an antibody.

36. The method of claim 31, wherein said detection agent is an aptamer.

37. The method of claim 31, wherein said response agent is an enzyme.

38. The method of claim 31, wherein said response agent is a fluorophore.

39. The method of claim 31, wherein said first binding moiety is an azide group.

40. The method of claim 31, wherein said first binding moiety is a streptavidin protein.

41. The method of claim 31, wherein said first binding moiety is an iodoacetyl group.

42. The method of claim 31, wherein said second binding moiety is an alkynyl group.

43. The method of claim 31, wherein said third binding moiety is an alkynyl group.

44. The method of claim 31, wherein said second binding moiety is a biotinyl group.

45. The method of claim 31, wherein said third binding moiety is a biotinyl group.

46. The method of claim 31, wherein said second binding moiety is a sulfhydryl group.

47. The method of claim 31, wherein said third binding moiety is a sulfhydryl group.

48. A method of making a complex for detecting analytes, said method including:
adding a linker-capture agent complex to a linker-detection agent complex,
wherein said linker-capture agent complex includes:
a first linker,
wherein said first linker includes a first binding moiety,
a capture agent; and
a fluorophore,
wherein said linker-detection agent complex includes:
a second linker,
wherein said second linker includes a second binding moiety,
a detection agent; and
a fluorophore,
wherein when said first binding moiety and said second binding moiety come into proximity with one another they form a chemical bond.

49. The method of claim 42, wherein said first linker is a nucleic acid.

50. The method of claim 42, wherein said first linker is a polyethylene glycol polymer.

51. The method of claim 42, wherein said second linker is a nucleic acid.

52. The method of claim 42, wherein said second linker is a polyethylene glycol polymer.

53. The method of claim 42, wherein said capture agent is an antibody.

54. The method of claim 42, wherein said capture agent is an aptamer.

55. The method of claim 42, wherein said detection agent is an antibody.

56. The method of claim 42, wherein said first binding moiety is a single stranded nucleic acid.

57. The method of claim 42, wherein said second binding moiety is a single stranded nucleic acid.

58. A method of detecting an analyte, said method including:
adding an analyte to a solution containing a capture agent and a detection agent,
wherein said capture agent and said detection agent are chemically bound to a solid support,
wherein said analyte forms a chemical bond to said capture agent,
wherein said analyte forms a chemical bond to said detection agent,
cleaving the bond between said detection agent and said solid support,
activating said response agent on said detection agent,
wherein said response agent produces a measurable signal in response to activation, and
measuring strength of said signal to detect the presence of said analyte.

59. The method of claim 52, wherein said capture agent is an antibody.

60. The method of claim 52, wherein said detection agent is an antibody.

61. The method of claim 52, wherein said capture agent is an aptamer.

62. The method of claim 52, wherein said detection agent is an antibody.

63. The method of claim 52, wherein said response agent is an enzyme.

64. The method of claim 52, wherein said response agent is a fluorophore.

65. The method of claim 52, wherein said solid support is a multi-well plate.

66. A method of detecting an analyte, said method including:
adding an analyte to a solution containing a capture agent and a detection agent,
wherein said capture agent and said detection agent are chemically bound to one another by a linker,
wherein said analyte forms a chemical bond to said capture agent,
wherein said analyte forms a chemical bond to said detection agent,
cleaving the linker between said capture agent and said detection agent,
activating said response agent on said detection agent,
wherein said response agent produces a measurable signal in response to activation, and
measuring strength of said signal to detect the presence of said analyte.

67. The method of claim 60, wherein said capture agent is an antibody.

68. The method of claim 60, wherein said capture agent is an aptamer.

69. The method of claim 60, wherein said detection agent is an antibody.

70. The method of claim 60, wherein said detection agent is an aptamer.

71. The method of claim 60, wherein said linker is a nucleic acid.

72. The method of claim 60, wherein said linker is a polyethylene glycol polymer.

63. The method of claim 60, wherein said response agent is a fluorophore.
